(19) United States
(12) Patent Application Publication
(10) Pub. No.: US 2025/0073417 A1
(43) Pub. Date: Mar. 6, 2025

(54) SYSTEMS, DEVICES AND METHODS FOR CALORIC AND GALVANIC VESTIBULAR STIMULATION POST ANESTHESIA ADMINISTRATION

(71) Applicant: Scion NeuroStim, Inc., Durham, NC (US)

(72) Inventors: Robert D. Black, Chapel Hill, NC (US); Lanty L. Smith, Durham, NC (US); Lesco L. Rogers, Raleigh, NC (US); Amanda B. Tucker, Cary, NC (US); Braden Parrish Garrison, Durham, NC (US)

(21) Appl. No.: 18/825,082

(22) Filed: Sep. 5, 2024

Related U.S. Application Data
(60) Provisional application No. 63/580,523, filed on Sep. 5, 2023.

(51) Int. Cl. A61M 21/00; A61N 1/04; A61N 1/08; A61N 1/20

(52) U.S. Cl. CPC A61M 21/00; A61N 1/0456; A61N 1/08; A61N 1/205; A61M 2021/0066; A61M 2021/0072; A61M 2021/0083; A61M 2210/0662

(57) ABSTRACT

A method for administering vestibular stimulation to a subject after anesthesia administration includes after reducing or ceasing administration of anesthesia, administering galvanic vestibular stimulation (GVS) to the subject; and reducing or ceasing administration of GVS at a time when an anesthetized state of the subject is reduced.

### CROSS-REFERENCE TO RELATED APPLICATION

The present application claims priority from and the benefit of U.S. Provisional Patent Application No. 63/580,523, filed Sep. 5, 2023. The disclosure of which is hereby incorporated herein by reference in its entirety.

### FIELD OF THE INVENTION

The present invention relates to vestibular stimulation, and in particular, to caloric and/or galvanic vestibular stimulation delivered after administration of anesthesia.

### BACKGROUND

Recovery from inhalational anesthetics is primarily a passive process initiated when administration ceases post-procedure. Clinical issues associated with delayed or inadequate anesthetic emergence include postoperative delirium, which occurs in 50% of elderly adults and 50-80% of children. Furthermore, surgeons, anesthesiologists, and nurses require additional time to ensure patients properly recover post-operatively, contributing to increased costs and capacity challenges. Electrical stimulation via implanted electrodes of the ventral tegmental area aids arousal from anesthesia in rats; however, this approach is not feasible to translate to humans. Solt K et al. “Electrical Simulation of the Ventral Tegmental Area Induces Reanimation from General Anesthesia,” Anesthesiology, 2014; 121(2): 311-319.

### SUMMARY

A method for administering vestibular stimulation to a subject after anesthesia administration includes after reducing or ceasing administration of anesthesia, administering galvanic vestibular stimulation (GVS) to the subject; and reducing or ceasing administration of GVS at a time when an anesthetized state of the subject is reduced.

### DESCRIPTION OF THE DRAWINGS

FIG. 1 is a schematic diagram of a device for galvanic vestibular stimulation (GVS) according to some embodiments.

FIG. 2 is a schematic of the device of FIG. 1 placed in an ear canal.

FIG. 3 is a perspective view of a controller and a headset according to some embodiments.

FIG. 4 is a front view of a user interface for the controller of FIG. 3.

FIG. 5 is an enlarged perspective view of the earpiece of the headset of FIG. 3.

FIG. 6 is a side view of earpiece and an ear according to some embodiments.

FIG. 7 is a perspective view controller according to some embodiments.

FIG. 8 is a side view of a stand for the controller according to some embodiments.

FIG. 9 is a perspective view of a stand for the controller according to some embodiments.

FIG. 10 is a headset for performing both GVS and caloric vestibular stimulation according to some embodiments.

### DETAILED DESCRIPTION

The present invention now will be described hereinafter with reference to the accompanying drawings and examples, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity.

### Definitions

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under.” The device may be otherwise oriented (rotated 90 degrees or at other orientations) and the spatially relative descriptors used herein interpreted accordingly. Similarly, the terms “upwardly,” “downwardly,” “vertical,” “horizontal” and the like are used herein for the purpose of explanation only unless specifically indicated otherwise.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present invention. The sequence of operations (or steps) is not limited to the order presented in the claims or figures unless specifically indicated otherwise.

The present invention is described below with reference to block diagrams and/or flowchart illustrations of methods, apparatus (systems) and/or computer program products according to embodiments of the invention. It is understood that each block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the function/act specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, the present invention may be embodied in hardware and/or in software (including firmware, resident software, micro-code, etc.). Furthermore, embodiments of the present invention may take the form of a computer program product on a computer-usable or computer-readable non-transient storage medium having computer-usable or computer-readable program code embodied in the medium for use by or in connection with an instruction execution system.

The computer-usable or computer-readable medium may be, for example but not limited to, an electronic, optical, electromagnetic, infrared, or semiconductor system, apparatus, or device. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory such as an SD card), an optical fiber, and a portable compact disc read-only memory (CD-ROM).

As used herein, the term “vestibular system” has the meaning ascribed to it in the medical arts and includes but is not limited to those portions of the inner ear known as the vestibular apparatus and the vestibulocochlear nerve. The vestibular system, therefore, further includes, but is not limited to, those parts of the brain that process signals from the vestibulocochlear nerve.

As used herein, the term “anesthesia” refers to a class of drugs used to anesthetize patients, for example, during surgery. Examples include inhaled agents, such as desflurane, enflurane, halothane, isoflurane, methoxyflurane, nitrous oxide, sevoflurane, and xenon. Intravenous agents may also be included in the term “anesthesia” and include propofol, etomidate, ketamine, barbiturates, and benzodiazepines.

After the administration of anesthesia or when the administration of anesthesia is reduced, subjects may show signs of arousal from the effects of anesthesia after a procedure. It should be understood that common metrics of observing a subject “waking” from the effects of anesthesia may be used to determine when GVS and/or CVS therapy may be reduced or stopped.

Embodiments according to the present invention may be used in animal and human subjects, but are primarily used in human subjects.

Recently Eisen et al. (Neuron, v. 112, 1-15, 2024) provided evidence that many anesthetics (including common inhalational anesthetics) act as GABA agonists and that this enhanced inhibition upsets dynamic stability in brain networks. This work is consistent with the concept in neuroscience of E-I balance, which posits that brain networks are active in a narrow range between excitation and inhibition. Hagihira (Br. J. Anesthesia, v. 115, suppl 1, i27-i31, 2015) shows the effects of isoflurane concentration on EEG spectra, which provides a separate perspective on the upset of dynamic stability during anesthesia. Another finding related in Eisen et al. is that primary sensory cortex responses to sensory stimuli are less affected by anesthesia than those in the higher cortex. The higher cortex shows a very delayed response to sensory stimuli during anesthesia. Emergence from anesthesia may be characterized by the re-establishment of E-I balance.

There are two primary ways by which E-I balance can be regained. The first is to clear the anesthetic molecules from GABA-ergic neurons where they act as agonists. Like tvCVS, tvGVS operating in the B wave range of frequencies may act to modulate cerebral blood flow (Black et al., JTEHM, 4:2000310, 2016) creating oscillations in flow, which will act to clear the anesthetic compound. tvGVS may also accelerate respiratory rate (Heckmann et al., Acta Neurol Scand, v. 100, 12-17, 1999), which also acts to clear inhalational anesthetics. The second way that tvGVS may act to regain E-I balance is through the unique innervation pattern of the vestibular sensory network (Black & Rogers, Front Syst Neurosci, v. 14:12, 2020). Starting from the vestibular nuclei in the brainstem, vestibular afferents connect with multiple brainstem structures, including the reticular activating system, and then the thalamic reticular nucleus (ventral thalamus) and the dorsal thalamus. From the thalamus, effectively all cortical regions receive vestibular input because there is no true central vestibular cortex and it is widely distributed. By contrast, the auditory cortex is very discrete and regionalized, even though the same cranial nerve (8th) transmits vestibular and auditory information. Subcortical structures (especially the brainstem) and the primary sensory cortices are, again, less impacted by anesthesia. The reticular activating systema and thalamic reticular nucleus are crucial in supporting conscious awareness. Voss et al. (Anesthesiology, v. 130, 1049-63, 2019) suggest that thalamocortical relays may be the likely targets of anesthetics versus the thalamus itself. This is consistent with the findings in Eisen et al. since both sets of authors identify cortical processing of sensory information as being highly degraded during anesthesia.

Accordingly and without wishing to be bound by any one particular theory, it may be concluded that vestibular sensory information not only creates activity in subcortical structures that support conscious awareness (like the RAS & TRN) but it is better able to reach sensory processing areas in the cortex, thereby working to re-establish E-I balance and foster the return of consciousness.

To reduce or overcome the challenges associated with delayed arousal from anesthesia, vestibular stimulation devices may be used. Although embodiments according to the present invention are described below with respect to in-ear GVS devices, it should be understood that any suitable GVS or CVS or combined GVS/CVS device may be used, including GVS that is administered at a different location (e.g., mastoid bone) or with different techniques, including single frequency techniques. In some embodiments, an improved, portable galvanic vestibular stimulation (GVS) device to enable safe and easy administration in postoperative care settings may be used; however, any suitable vestibular stimulation device may be used, including caloric vestibular stimulation devices (CVS), GVS devices, or combined GVS and CVS devices to administer GVS and CVS either simultaneously or successively.

Non-limiting examples of devices that may be used to deliver GVS and/or CVS after anesthesia may be found in U.S. Pat. Nos. 9,532,900, 10,945,879, and 11,794,010, the disclosures of which are incorporated by reference herein in their entirety.

In some embodiments, GVS devices may be used for post-anesthesia arousal that 1) use in-the-ear electrodes; 2) have a headset-like design that exerts uniform pressure on the tissue of the ear canal, facilitating consistent contact impedance and operation in a constant-current mode; 3) require lower voltages due to the application of a high-frequency carrier (>1 kHz) to allow for low-impedance passage through the skin and improved safety. This device design may reduce the need for special preparation on the part of clinical staff, thereby facilitating use in postoperative settings.

In particular, a modulated electrical signal may be transmitted through the skin lining the ear canal to stimulate the vestibular system of the subject, e.g., as described in U.S. Pat. No. 10,945,879. The skin may provide an electrical resistance in the electrical path between the electrode and the vestibular system. The electrical resistance of the skin may be generally inversely proportional to the frequency of the electrical signal. Thus, in order to stimulate the vestibular system at lower frequencies, a waveform of larger amplitude may be required than a waveform at higher frequencies. The larger amplitude may not be desired as the subject may experience discomfort, pain, and/or physical damage based on the large voltage. However, the higher frequencies may not induce the desired effects of galvanic vestibular stimulation. For example, some diagnostic and/or therapeutic uses of galvanic vestibular stimulation desire stimulation at lower frequencies. In some embodiments of the present invention, a modulation scheme is provided that generates an electrical signal with a higher frequency to produce the lower impedance and that stimulates the vestibular system at a lower frequency, and the lower frequency may be desirable for use after the administration of anesthesia to more quickly arouse a patient from anesthesia.

For example, the modulation scheme may provide a repeating series of spaced-apart packets of electronic pulses. The electronic pulses within the packets may be closely separated in time to provide the higher frequency and, thus, to produce the lower impedance that permits transmission through the skin. One or more parameters may be modulated according to a lower frequency. For example, one or more of the quantity of the plurality of pulses within ones of the plurality of packets of pulses, the width in time of the plurality of electrical pulses within ones of the plurality of packets of pulses, the amplitude of the plurality of pulses within ones of the plurality of packets of pulses, the separation in time between adjacent ones of the plurality of pulses within ones of the plurality of packets of pulses, and the separation in time between adjacent ones of the plurality of packets of pulses may be modulated. The vestibular system may be stimulated based on the lower frequency, which may be referred to herein as the “effective GVS frequency” or “effective frequency.” For example, the lower frequency modulation may entrain brainwaves based on the low frequency of the modulation. Thus, the modulation scheme may produce the lower impedance based on the higher frequency of the pulses within a packet and stimulate the vestibular system based on the lower frequency of the modulation.

In some embodiments, the need to clean or even apply abrasion to the skin can be reduced or eliminated. The electrode may be held in position in the ear canal by an earpiece or headset. In some embodiments, a conductive gel may be applied to the ear or on the electrode to increase conductivity through the skin.

A non-portable benchtop implementation of GVS architecture has been developed, successfully establishing the basis for the creation of a portable GVS headset prototype. Accordingly, GVS may be used in accelerating emergence from anesthesia in animal and human models.

In some embodiments, in-ear electrode placement for GVS and a modulated current source are used. Whereas current laboratory based GVS systems deliver current through electrodes placed onto the mastoid bone, a GVS car-insert design makes use of conductive earpieces that enter the exterior ear canal, increasing proximity to vestibular organs and reducing the power needed to perform GVS. In addition, traditional GVS systems placed on the mastoid bone involve careful placement of flexible electrodes and a careful cleaning of the electrode placement cite for low contact impedance. However, GVS simulation may utilize a high-frequency carrier and envelope modulation to create an effective GVS frequency in the 0.001-100 Hz range, the conventional frequency range of most GVS research, with an exceptionally low applied voltage of <10V to reduce the need to clean the site of electrode placement for ease of use. Specific effective frequency ranges of 0.008-0.05 HZ or 0.5-2.0 HZ may be used as described in additional detail herein. Portable GVS headsets may be used for either civilian or military applications. Detailed descriptions of high-frequency carrier and envelop modulation for GVS frequencies are described in U.S. Pat. No. 10,945,879.

Without wishing to be bound by any particular theory, in some cases, vestibular stimulation may be verified by observing induced head movements resulting from GVS stimulation and the frequency of movements may match the frequency of the applied GVS stimulus. This effect is thought to be a compensatory response to biasing of the otolith structures that sense the orientation of the body in a gravitational field. The applied frequency can be in a range to which the body can respond: too fast and the muscles can't follow the motion and too slow and the motion will be hard to observe. In some embodiments, a range of 0.5-2.0 Hz may be used to produce induced head movements. Saabani et al. Data on galvanic-evoked head movements in healthy and unilaterally labyrinthectomized rats. Data Brief. 2016; 9:338-344 demonstrate the GVS evoked head movements and provide evidence that it is indeed a vestibular reaction versus a more generalized response to electrical stimulation of the head. This effect has been demonstrated in preliminary tests with rats as they emerge from isoflurane anesthesia.

Accordingly, a GVS stimulus that utilizes a frequency range for producing induced head movements may be used to select a current value for administering GVS. For example, administering GVS may include the following. First, an electrical signal may be administered in which the electrical signal comprises a high-frequency carrier and envelop modulation that is configured to create an effective GVS frequency in a range of 0.5 to 2.0 HZ. Next, the current of the electrical signal may be increased until head movements are observed in the subject to determine a treatment current. After head movements are observed in the subject, the frequency of the electrical signal may be modified so that the electrical signal is configured to create an effective GVS frequency in a range of 0.008 to 0.05 HZ that is administered at the treatment current. For example, the user (or health care professional) may enter a user input when the user observes that head motion is induced to initiate the controller to set the treatment current as the administered current. The electrical signal may be administered with an effective GVS frequency in the range of 0.008 to 0.05 HZ at the treatment current until the time when the anesthetized state of the subject is reduced. Again, without wishing to be bound by any particular theory, the frequency of the effective GVS signal in the range of 0.008 to 0.05 HZ is presently believed to be effective for exciting cerebral blood flow oscillations (i.e., a B wave range of frequencies), while the range of 0.5 to 2.0 Hz may be effective for inducing head movement. Therefore, effective GVS frequencies for inducing head movement (e.g., 0.5 to 2.0 HZ) may be used to determine a treatment current for administering an effective GVS frequency at a constant treatment current to induce cerebral blood flow oscillations (e.g., 0.008 to 0.05 HZ).

In addition, safety thresholds may be maintained for the treatment current and the voltage of the GVS signal by the controller, for example, such that the treatment current may be less than 5 mA and the voltage may be less than 10 volts. In some embodiments, a constant current mode is used.

In some embodiments, the use of a high frequency carrier voltage as described in U.S. Pat. No. 10,945,879 reduces or eliminates the need for special electrode preparation. The device may be used in a hospital, for example, mounted on an IV-pole. Rechargeable batteries may be used so that the device may be mobile without the need for a power plug when in use; however, power from an outlet may also be used. In military applications, the design may be more rugged, such as being sealed from dust or to incorporate water proofing to withstand harsh environments. The frequencies of treatment may be pre-selected and current levels may be adjustable within a predetermined limit, typically under 2 mA.

A disposable set of electrodes may be used that are sized and configured to fit into the car canal, such as an “ear bud” with a metallic foil to contact the wall of the auditory canal to deliver GVS. An impedance measurement circuit may be used to verify that the contact between the electrode (metallic foil) and the ear canal are secure and may support the desired current flow. A light/tone indicator may be used so that the user can receive feedback when the electrode properly contacts the ear canal.

The delivery of GVS is typically for five minutes or less, but in some embodiments, may last up to 15 minutes.

A general protocol for administering vestibular stimulation post anesthesia is as follows. The system may be charged or otherwise readied, such as inserting the GVS (and/or CVS) earpieces into the patient's ear canal, as the end of a procedure involving anesthesia is approaching and the amount of anesthetic is being tapered downward. The proper placement of the earpieces may be affirmed by determining the impedance value between electrodes in the car. In some embodiments, proper placement of the earpieces may be verified before anesthesia is stopped. Once anesthesia is greatly reduced or stopped, GVS stimulation may be initiated and continue until the patient is clearly awake and responsive. In some embodiments, however, GVS and/or CVS treatment may be continued for a period of time after the patient has emerged from anesthesia, for example, to reduce the likelihood of sequela such as delirium. Such aftercare may further reduce neuroinflammation.

The potential benefits of vestibular stimulation to aid anesthesia arousal drive the need for a device that can be used safely and easily in post-operative recovery settings. A portable, user-friendly GVS device may be designed to aid anesthesia arousal and reduce the amount of time in post-operative recovery.

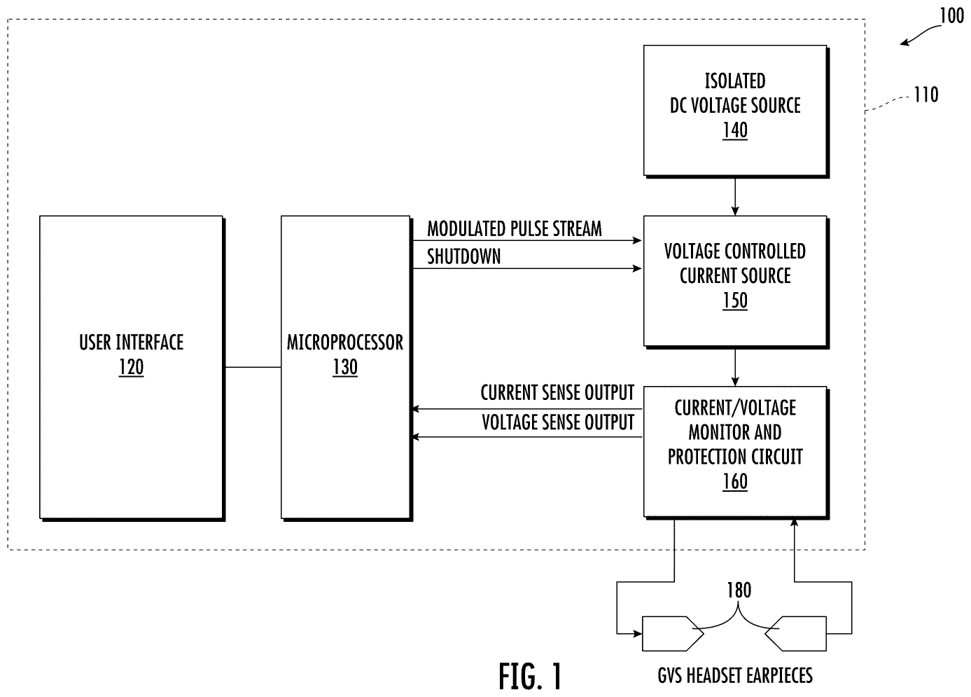

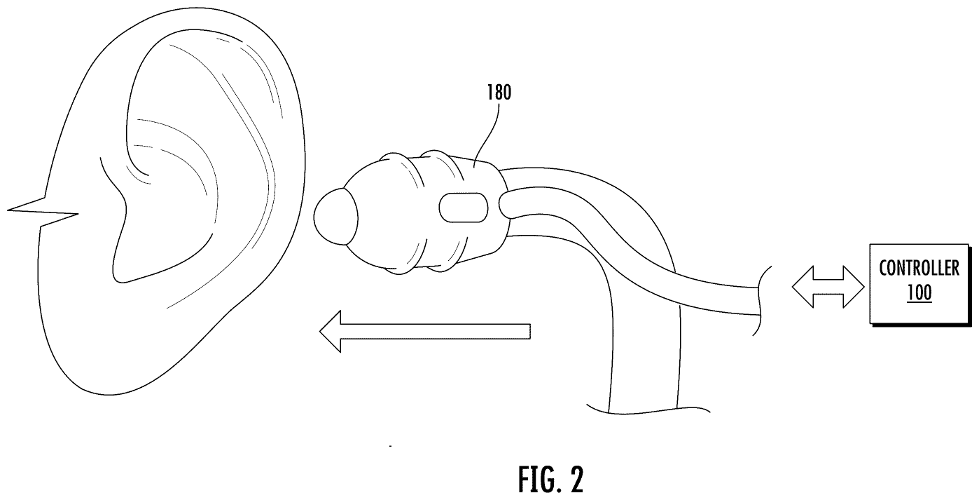

As illustrated in FIGS. 1-2, an in-ear stimulation device 100 includes a controller 110 that is in communication with two GVS headset earpieces 180. The controller 110 includes a user interface 120, a central processing unit (CPU) or microprocessor 130, an isolated DC voltage source 140, a voltage controlled current source 140, and a current/voltage monitor and protection circuit 160.

In some embodiments, a touchscreen display may be used as part of the controller 110 as the user interface 120. The display may include information displayed to the user, such as the battery status, electrode impedance indicator (which may be binary, e.g., good/bad), the current level (with optional adjustment controls), and a clock or timer.

Although in some embodiments, electrodes may be positioned on the mastoid bone behind the ear, using an electrode configured to be positioned in the ear canal with a metal electrode earpiece may reduce the need for specialized placement of adhesive electrodes, allowing easy application of the device (e.g., similar to a music headset). This will also increase the proximity of the electrodes to the vestibular organs, and in some embodiments, may reduce the power needed to perform GVS.

The monitor and protection circuit 160 may provide a means for the microprocessor 130 to measure an instantaneous electrical current and voltage being applied during the treatment and limit the maximum voltage and current that is applied. This approach may allow the device 100 to adjust for changes in the skin impedance of the patient.

The GVS device 100 may be designed for increased patient safety. The use of an isolated DC voltage source 140 to power the current source 150 may limit the maximum potential that can be applied between the GVS device earpieces 180. The isolated DC voltage source 140 may also reduce or prevent electrical current from other circuits in the device 100 from leaking to the patient. The current source maximum from the isolated DC voltage source 140 to the voltage controlled current source 150 may be adjustable, but in some embodiments, is about 2 mA. The monitor and protection circuitry 160 may provide hardware protection circuits as well as outputs to the microprocessor 130, enabling the firmware to provide safety monitoring. The microprocessor 130 may monitor the average current and voltage being applied and shut down the treatment if the current or voltage exceed set safety limits. The use of pulse-width modulation of the carrier signal may also provide additional safety features because pulse-width modulation of the carrier signal may limit the total power applied to the patient. For instance, a 20% duty cycle means that current is not present for 80% of the stimulation period, whereas some past investigations using DC GVS reported a risk of burns to patients because of the continuous application of current.

In some embodiments, the system 100 utilizes a low voltage (<10V) to generate an effective GVS frequency in the 0.001-100 Hz range through the combination of a high-frequency carrier and envelope modulation, wherein specific ranges of 0.008-0.05 HZ and 0.5-2.0 HZ may be used. To implement this approach, a current pulse (using a constant-current circuit) that represents the carrier frequency is applied to the GVS electrodes. The amplitude of that current pulse is then modulated at the desired “working” GVS frequency, which will be in the low-Hz or even sub-Hz range. Thus, the net effect is to achieve low impedance coupling with the GVS device and still have conventional GVS frequencies for neuromodulation, lowering the overall power applied to the device and patient.

Accordingly, the system 100 encapsulates GVS delivery within a portable, comfortable, and user-friendly headset using earpieces 180. The headphone-like design of the GVS device 100 is both familiar to users and functional: the earpieces 180 may exert uniform or substantially uniform pressure on the tissue of the ear canal, facilitating both constant contact impedance and operation in a low-voltage, modulated current mode. Further, its portable nature is convenient for post-operative recovery settings.

These potential advancements in GVS technology may improve the safety and usability of GVS-based treatment, facilitating a shift to post-operative applications of GVS and case of use in the post-operative settings.

### Portable Version of the Hardware/Software.

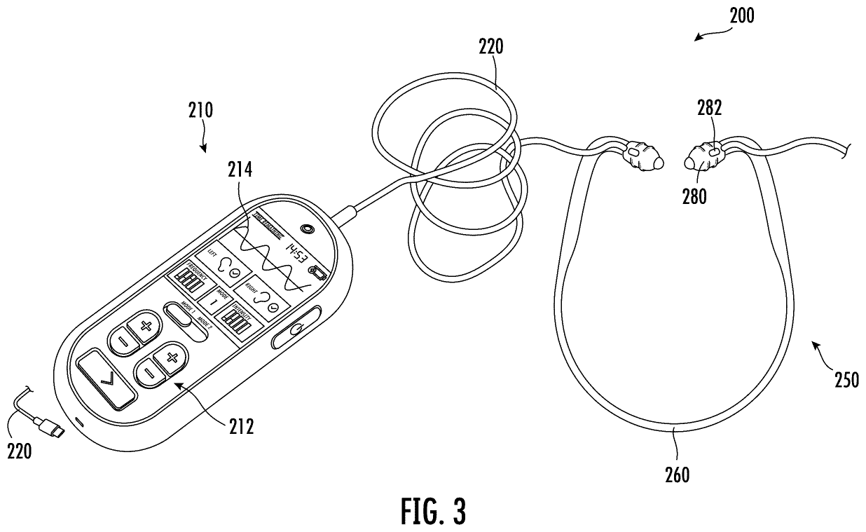

As illustrated in FIG. 3, a mobile device 200 may include a controller 210 and a headset 250. The controller 210 includes a user interface 212 and display for controlling the headset 250 and a power supply 220. The headset 250 is connected to the controller 210 with a cable 220 configured to communicate and deliver electrical signals from the controller 210 to the headset electrode 282. The cable 220 may be flexible.

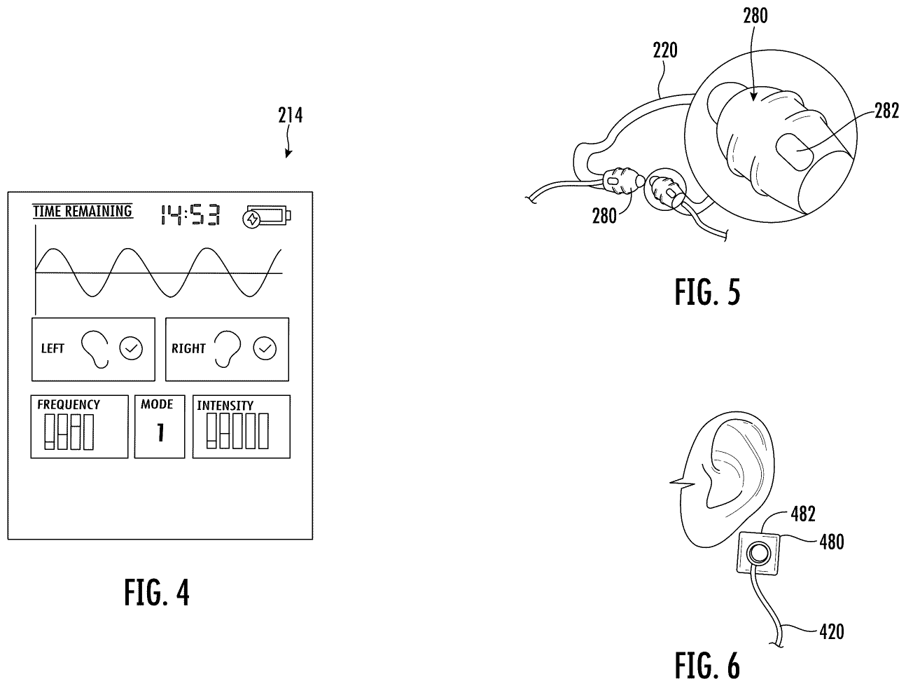

As illustrated in FIGS. 3 and 4, the user interface 212 may include controls for controlling an amount of time for applying GVS stimulation, starting and stopping GVS stimulation, and/or controlling an amount or frequency of stimulation. The interface may also allow for rapid switching between frequency ranges, for example 0.5-2.0 Hz for setting the current level based on induced head flexion and 0.008-0.05 Hz for the extended stimulus period to accelerate emergence from anesthesia. The interface may also control the amount of current applied within set limits. The GVS stimulation may be set in a constant current mode. The interface may also have indicator LED's or other indicators, such as a visual indicator on the user interface 212, to confirm that a target impedance range is achieved when the electrodes are placed.

As illustrated in FIGS. 3 and 5, the headset 250 includes earpieces 280 that are connected by a headband 260. GVS electrodes 282 are positioned on the earpieces 280. In this configuration, the headset 250 enables the placement of in-the-ear electrodes 282 with earpieces 280 that are positioned in the ear canal. In some embodiments, the earpieces 280 may exert substantially uniform pressure on the tissue of the ear canal for positioning the electrodes 282 in contact with ear canal tissue. This arrangement will allow for increased proximity of electrodes 282 to the vestibular organs, consistent contact impedance for the electrodes 282 within the ear, as well as user comfort. The application of a high-frequency carrier may reduce or eliminate the need for special skin preparation, since the high-impedance dead skin layer acts as a capacitive, not a resistive, element. The high frequency carrier may then be envelope modulated to achieve the desired GVS operating frequency, e.g., 0.5-2.0 Hz or 0.008-0.05 Hz. The electrodes 282 may be any suitable conductive electrode, including electrodes that metal electrodes that have a conductive coating for increased comfort during use.

Although the headset 250 is illustrated with respect to the earpieces 280 and electrodes, it should be understood that any suitable earpiece may be used, for example, as illustrated with respect to the earpiece 480 and electrode 482 in FIG. 6. The electrode 482 is connected directly to a cable 420, which in turn is in communication with a controller (not shown).

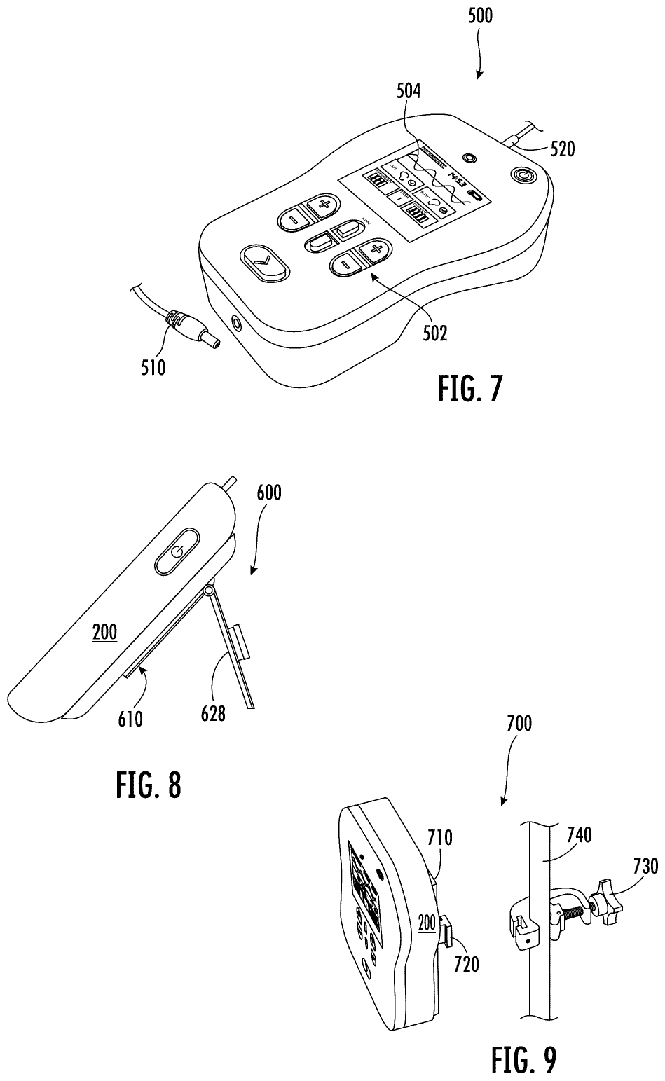

In addition, alternative configurations of a controller may be used. As shown in FIG. 7, a controller 500 includes a user interface 502 and display 504, which is configured to connect to a power source 510 and a headset connector cable 520.

The mobile GVS device may be connected to a stand or other device for increased mobility and case of use. As shown in FIG. 8, a stand 600 includes a base 610 for holding the controller 200 and a kickstand 620 for positioning the controller 200 in an angled positioned so that the user can easily view and operate the controller 200. As shown in FIG. 9, in some embodiments, the controller 200 may be connected to a stand 700 that connects to a pole, such as an IV pole 740. The stand 700 includes a base 710 that holds the controller 200 and includes a connector 720. The connector is configured to connect to a clamp 730 on an IV pole 740. In this configuration, the controller 200 may be provided on an IV pole 740, which is generally provided in clinical settings for case of use.

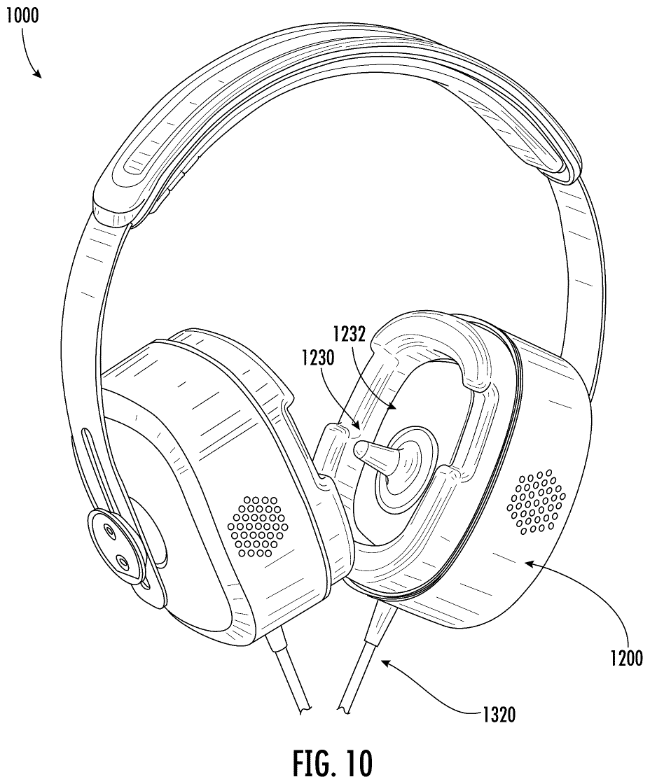

It should be understood that GVS delivery electrodes may be provided in a combination GVS and caloric vestibular stimulation (CVS) device. Previously, a clinically tested and FDA-cleared neuromodulation device for delivering caloric vestibular stimulation (CVS) with a similar headset design has been developed. See U.S. Pat. No. 11,896,826. As illustrated in FIG. 10, the in-ear stimulation device 1000 includes earcups 1200 having a housing 1300 for holding the earpieces 1230 in the ear canals of a user, cushioning members or foam padding 1310, and connectors 1320 for connecting to a controller, such as the controller 200.

As described in U.S. Pat. No. 11,896,826, the earpiece 1230 are suspended on a specialized elastic diaphragm 1232. The device 1000 holds the outer ear cups 1200 against the head, but the earpieces 1230 are free to move on the elastic diaphragm 1232 so that the angle of the earpiece 1230 adjusts to the ear canal. The diaphragm 1232 also acts as a spring to keep the earpiece fully inserted into the ear canal during use, allowing for comfort for the user as well as good contact consistency for the earpiece 1230. This design may be implemented for the portable GVS device, with the earpiece 1230 simultaneously serving as the electrode, such as the electrode 180 of FIG. 1. In particular embodiments, the device 1000 may also incorporate a thermoelectric (TED) device for heating and cooling the earpiece to combine GVS and CVS as controlled by the controller 100. In this configuration the metal earpieces 1230 may be attached to a thermoelectric (TED) device for providing a CVS waveform and also provide a conductive surface for performing GVS. Combined GVS and CVS systems are further described in in U.S. Pat. No. 10,945,879.

As discussed above, the hardware and firmware architecture that will be utilized within the portable device is shown in the schematic diagram of the hardware of the device above. Firmware will generate a pulse-width modulated carrier waveform (1-10 KHz) that will be envelope modulated to ˜1 Hz. This waveform will be delivered to the patient in a constant current mode, which will ensure a consistent GVS stimulus. The high frequency carrier makes the electrode-skin contact impedance primarily capacitive, thus making the impedance only weakly dependent on the DC contact resistance.

In some embodiments, the anesthetized state of the subject is determined by a health care provider, who ceases administration of the vestibular stimulation at a time when the subject shows a reduced anesthetized state. However, it should be understood that the period of vestibular stimulation may be predetermined based on an average time for a subject to have a reduced anesthetized state after administration of GVS, such as less than five minutes or up to 15 minutes. For outpatient surgery, i.e., with no overnight stay in a hospital, patients are typically transported from an operating room to a post-anesthesia care unit (PACU) and then to a more general recovery room. If the surgery and/or anesthesia is more significant, patients may be transported from an operating room to an intensive care unit (IC) or a PACU and then to a hospital room for an overnight stay. In some embodiments, administration of vestibular stimulation (GVS and/or CVS) may be initiated directly in the operating room with a portable vestibular stimulation unit as described herein that is moved together with the patient.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

What is claimed is:

1. A method for administering vestibular stimulation to a subject after anesthesia administration, the method comprising:
after reducing or ceasing administration of anesthesia, administering galvanic vestibular stimulation (GVS) to the subject; and
reducing or ceasing administration of GVS at a time when an anesthetized state of the subject is reduced.

2. The method of claim 1, wherein administering galvanic vestibular stimulation (GVS) to the subject comprises positioning a pair of electrodes on a subject in a position configured to provide galvanic vestibular stimulation.

3. The method of claim 2, wherein administering galvanic stimulation (GVS) comprises inserting a first earpiece having a first electrode thereon into one of the ear canals the subject and inserting a second earpiece having a second electrode thereon into another of the ear canals of the subject, and delivering an electrical signal to the ear canals of the subject to thereby provide the CVS.

4. The method of claim 3, wherein administering galvanic stimulation (GVS) further comprises:
administering the electrical signal such that the electrical signal comprises a high-frequency carrier and envelop modulation that is configured to create an effective GVS frequency in a range of 0.5 to 2.0 HZ;
increasing a current of the electrical signal until head movements are observed in the subject to determine a treatment current;
after head movements are observed in the subject, modifying the electrical signal such that the electrical signal is configured to create an effective GVS frequency in a range of 0.008 to 0.05 HZ and at the treatment current; and
continuing administering the electrical signal with an effective GVS frequency in the range of 0.008 to 0.05 HZ at the treatment current until the time when the anesthetized state of the subject is reduced.

5. The method of claim 4, wherein the electrical signal comprises a voltage of less than about 10 volts and the treatment current is a constant current below 5 mA.

6. The method of claim 3, wherein the first and second electrodes are connected to a controller configured to provide the electrical signal.

7. The method of claim 1, wherein administering GVS to the subject is performed for fifteen minutes or less.

8. The method of claim 1, further comprising administering caloric vestibular stimulation (CVS) to the subject; and reducing or ceasing administration of CVS at the time when the anesthetized state of the subject is reduced.

9. The method of claim 8, wherein administering CVS is performed simultaneously with administering GVS.

10. The method of claim 9, further comprising providing a conductive earpiece and the GVS and CVS are administered simultaneously via the conductive earpiece.

11. The method of claim 1, further comprising applying a conductive gel to the ear or on the electrode to increase conductivity through the skin.

12. An in-ear stimulation device for administering vestibular stimulation to the ear canal of a subject, comprising:
(a) first and second earpieces configured to be insertable into the ear canals of the subject;
(b) first and second electrodes on respective ones of the first and second earpieces and configured to deliver a galvanic vestibular stimulation to the ear canal;
(d) a controller in communication with the first and second electrodes, the controller configured to generate a control signal to control an electrical signal delivered to the ear canal, to administer the electrical signal when the administration of anesthesia is reduced and to cease administration of the electrical signal at a time when an anesthetized state of the subject is reduced.

13. The device of claim 12, wherein the controller is configured to generate the control signal to
administer the electrical signal such that the electrical signal comprises a high-frequency carrier and envelop modulation that is configured to create an effective GVS frequency in a range of 0.5 to 2.0 HZ;
increase a current of the electrical signal;
accept a user input indicating that head movements are observed to determine a treatment current;
when the user input is received, modify the electrical signal such that the electrical signal is configured to create an effective GVS frequency in a range of 0.008 to 0.05 HZ at the treatment current; and
continuing to administer the electrical signal with an effective GVS frequency in the range of 0.008 to 0.05 HZ at the treatment current until the time when the anesthetized state of the subject is reduced.

14. The device of claim 13, wherein the electrical signal comprises a voltage of less than about 10 volts and the treatment current is a constant current less than 5 mA.

15. The device of claim 12, wherein the controller is configured to administer GVS to the subject for fifteen minutes or less.

16. The device of claim 12, further comprising a thermoelectric device connected to the first and second electrodes and configured to administer a caloric vestibular stimulation (CVS) to the subject, wherein the controller is configured to reduce or ease administration of CVS at the time when the anesthetized state of the subject is reduced.

17. The device of claim 16, wherein the controller is configured to administer CVS simultaneously with administering GVS.

18. The device of claim 12, further comprising a conductive gel on the first and second electrodes to increase conductivity through the skin.
